(19) United States
(12) Patent Application Publication
(10) Pub. No.: US 2006/0036319 A1
(43) Pub. Date: Feb. 16, 2006

(54) Cosmetic ophthalmic surgical conformer

(76) Inventor: Paul Theodore Finger, New York, NY (US)

Correspondence Address: William B. Gowanlock, 16 Town Crier Lane, Madison, CT, 06443, US

(21) Appl. No.: 10/917,589

(22) Filed: Aug. 13, 2004

(51) Int. Cl. A61F 2/14

(52) U.S. Cl. 623/6.64; 623/4.1

(57) ABSTRACT

A cosmetic ophthalmic surgical conformer for use in such surgical ophthalmic procedures as evisceration, enucleation, and exenteration. The cosmetic ophthalmic surgical conformer provides a prosthetic device with at least one eye representation for post-operative use extending up until the time that an artificial eye is available.

### FIELD OF THE INVENTION

The present invention relates generally to prosthetic devices and more specifically to ophthalmic surgical conformers that are used post-operatively in such ophthalmic procedures as evisceration, enucleation, and exenteration.

### BACKGROUND

The loss of sight in an eye is a traumatic, life-altering event which can occur for any number of reasons, such as trauma or disease. When this loss is accompanied by the removal of all or a portion of a patient's eyeball from an eye orbit, there is the additional trauma of loss of normal appearance, or disfigurement.

Surgical procedures that remove a portion, or all, of the eyeball and surrounding tissues within the orbit include evisceration, enucleation and exenteration. As these surgical procedures remove some, or all, of the eyeball and surrounding tissues, they create a volume in the orbit. The size of the volume depends on the procedure. For example, the eyeball can be broadly considered to comprise a sclera and a cornea that define a capsule that contains, among other structures, the uvea, retina, lens and vitreous. In an evisceration procedure, the cornea and the structures within the sclera are removed, leaving what is termed a scleral envelope. The enucleation procedure additionally removes the sclera and a portion of the optic nerve (a nerve connected to the eyeball a portion of which is within the orbit). This leaves in the orbit post-operative tissues, such as the rectus muscles, orbital fat and a residual envelope of fibrous connective tissue, termed Tenon's capsule. The exenteration procedure where the eyelids are spared, removes the remaining tissues in the orbit. In all these surgical procedure, the normal outward appearance of the eye is lost. Therefore, after such procedures as evisceration, enucleation, or exenteration, patients typically desire reconstruction to restore a more normal eye appearance.

Generally the restoration to create a more normal eye appearance employs an implant in combination with an artificial eye. The implant and artificial eye augment the volume created during the surgical procedure.

For an evisceration or enucleation surgical procedure, the implant is surgically placed within the scleral envelope or Tenon's capsule. This layer is then closed to prevent migration of the implant. After placement, conjunctiva (a membrane that covers most of the eyeball with a portion that is located between the back of the eyelids and the eyeball that during the procedure has had an incision made therein to gain access to the eyeball) is pulled over the implant and sutured back together thereby further securing the implant in the scleral envelope or Tenon's capsule. An ophthalmic surgical conformer (see Integrated Orbital Implants, Inc., Conformers, Part No. I00043) is then immediately placed over the conjunctiva in the eye socket proximate to the backside of the eyelids. After a healing period of weeks, a mold for an artificial eye will be made, and then some time later the artificial eye will be manufactured and fitted, which completes the augmentation of the volume, i.e., restoration.

In an exenteration procedure where the eyelids are retained, the implant, which is a dermis-fat graft, is placed within the orbit. The ophthalmic surgical conformer is placed between the graft and the eyelids to keep these tissues from adhering one to the other.

The ophthalmic surgical conformer is a temporary, transparent prosthetic device. It is recognized by the profession as separate and distinct from an artificial eye or even other conformers used in other ophthalmic surgical procedures, e.g., reconstruction due to congenital anophthalmia.

As mentioned above, the mold for the artificial eye is not taken until after a healing period. This healing period allows swelling, which naturally results from the surgical placement of the implant in the scleral envelope, Tenon's capsule or orbit, to subside. Due to this swelling, the volume has an initial residual volume into which the ophthalmic surgical conformer is placed that has a contour and volume that is different from a final residual volume into which the artificial eye will eventually be fitted and placed.

The ophthalmic surgical conformer has a therapeutic shape that allows it to fit into the initial residual volume, which is smaller than the final residual volume, and to act as part of a pressure system that maintains the shape and position of residual tissues, such as those in the conjunctiva. Pressure in the pressure system results from a monocular pressure dressing applied immediately post-operatively over the eye on the outside of the eyelids as firmly as possible to minimize swelling. It is the monocular pressure dressing that provides a force on the ophthalmic surgical conformer thereby permitting the ophthalmic surgical conformer to shape and position the residual tissues. This pressure is also thought to assist in preventing post-surgical hemorrhaging, as well as in maintaining the shape of the underlying tissues.

While the monocular pressure dressing is applied, the ophthalmic surgical conformer as well as the result of the surgical procedure is not viewable by an observer. Pressure, however, is only needed for a limited time after surgery, generally a matter of days. Therefore within days after surgery, the monocular pressure dressing is removed revealing to observers the missing eye. Unfortunately, it will be weeks, generally six to eight, until the final residual volume forms and can tolerate the molding procedure involved in making an artificial eye. Then additional time is required for the manufacturing of the artificial eye.

During the period after removal of the monocular pressure dressing and before the artificial eye is available and installed, the patient generally employs such items as sunglasses or an eye patch to obscure the view by observers of the eye socket. However, sunglasses appear out of place at certain times, particularly in the evening or inside. Eye patches also present issues that make them a less than desirable solution.

What is needed in the art is a cosmetically appealing prosthetic device for the period immediately after pressure bandage removal until an artificial eye is available for use.

### SUMMARY OF THE INVENTION

This invention in one aspect is a cosmetic ophthalmic surgical conformer. The cosmetic ophthalmic surgical conformer has at least one eye representation that is visible to an observer. The eye representation can include, but is not limited to, a pupil, an iris, a sclera and episcleral blood vessels.

These and other features, aspects, and advantages of embodiments of the present invention will become apparent with reference to the following description in conjunction with the accompanying drawings. It is to be understood, however, that the drawings are designed solely for the purposes of illustration and not as a definition of the limits of the invention.

### BRIEF DESCRIPTION OF THE DRAWINGS

FIG. 1 is a front view of the cosmetic ophthalmic surgical conformer of the present invention.

FIG. 2 is a side sectional view of the cosmetic ophthalmic surgical conformer depicted in FIG. 1.

FIG. 3 is a top sectional view of the cosmetic ophthalmic surgical conformer depicted in FIG. 1.

FIG. 4 is a series of side sectional views of cosmetic ophthalmic surgical conformers of the present invention depicting various schemes for incorporating at least one eye representation therein.

FIG. 5 is a side sectional view of an implant and the cosmetic ophthalmic surgical conformer of the present invention positioned in an eye socket.

FIG. 6 is a side sectional view of an implant and an artificial eye positioned in the same eye socket depicted in FIG. 5.

### DETAILED DESCRIPTION

As shown in FIGS. 1-3, the cosmetic ophthalmic surgical conformer 10 has a member 12 with which is associated at least one eye representation, collectively identified by reference number 14 and followed by a lower case letter, such as “a,” to identify a specific representation, defining a body 16. The at least one eye representation 14 may be a pupil representation 14a. Similarly, the at least one eye representation 14 may be an iris representation 14b. Additionally, the at least one eye representation 14 may be a sclera representation 14c, or at least one episcleral blood vessel representation 14d. It should also be appreciated that the above list of at least one eye representation 14 is not exclusive and that any combination of the above identified eye representations 14a, 14b, 14c, and 14d could be used on a single cosmetic ophthalmic surgical conformer 10.

In a profile of the body 16, the profile is generally egg-shaped. Additionally, it has a thickness t between a convex surface 18 opposed to a concave surface 20, which is substantially constant.

The at least one eye representation 14 can be as realistic as one desires. As a general matter, however, the cosmetic ophthalmic surgical conformer 10, just as the ubiquitous ophthalmic surgical conformer, should be an off-the-shelf item, not custom made for the patient. Therefore, it is anticipated that at least one eye representation 14 will be made in standard configurations and the “best match” chosen by a surgeon.

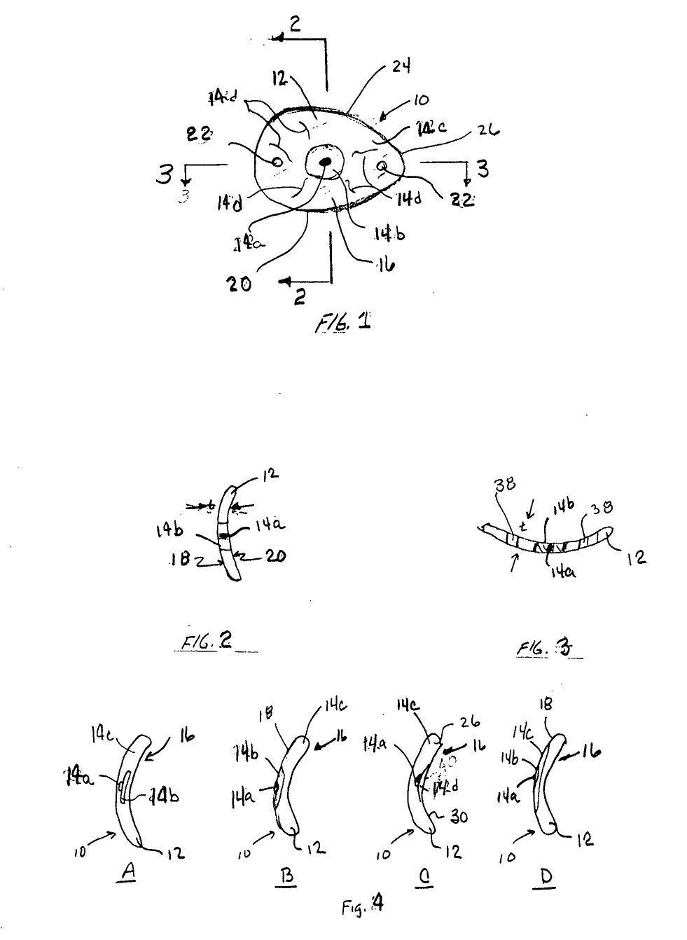

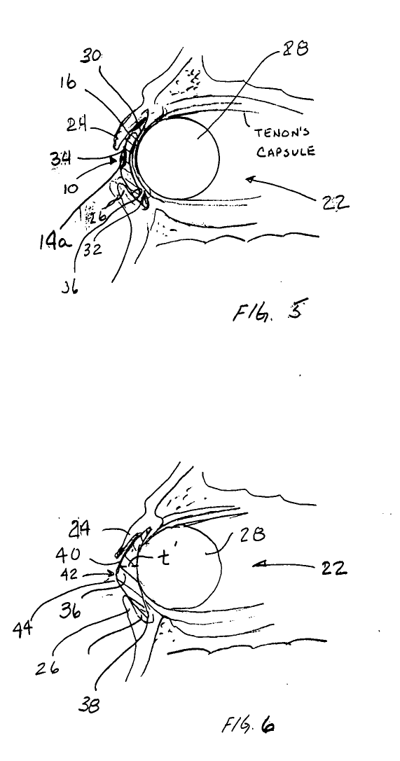

Referring to FIG. 4 A-D, the at least one eye representation 14 can be associated with the member 12 of the cosmetic ophthalmic surgical conformer 10 in any number of ways thereby defining the body 16. The at least one eye representation 14 can encapsulated within the member 12 or on the surfaces of the member. As shown in FIG. 4A, the pupil representation 14a and the iris representation 14b are solid bodies placed within the member 12. FIG. 4B shows the pupil representation 14a and the iris representation 14b as bodies defining a portion of the convex surface 18 of the cosmetic ophthalmic surgical conformer 10. FIG. 4C shows the pupil representation 14a and the iris representation 14b on the concave surface 20. Episcleral blood vessels (not shown) could similarly be added to the cosmetic surgical conformer 10. FIGS. 4A-C show the situation where the sclera representation 14c could be a coloring added to the member 12. Where coloring is used, the member 12 and the body 16 may be coincident. The coloring, however, need not extend to the surface of the body 16. FIG. 4D shows a pupil representation 14a, an iris representation 14b and a sclera representation 14c that has essentially been painted on or is a decal that has been placed on the convex surface 18 of the member 12 therefore defining the body 16.

FIG. 5 shows a cross-sectional view of an eye socket 22 post-operatively after enucleation of an eyeball (not shown). The cosmetic ophthalmic surgical conformer 10 is positioned in the eye socket 22 between an upper eyelid 24 and a lower eyelid 26, i.e. the eyelids 24, 26, and an implant 28, which is positioned in Tenon's capsule, in an initial residual volume 30. Conjunctiva 32 is positioned between the cosmetic ophthalmic surgical conformer 10 and the implant 28. It should be appreciated that if the at least one eye representation 14 is encapsulated within the member 12, the materials from which the at least one eye representation are made will not come in contact with the post operative tissues, e.g., Tenon's capsule.

The contour of the initial residual volume 30 is defined by the conjunctiva 32 and backsides 34, 36 of the eyelids 24, 26 and is a function of any swelling resulting from the surgical procedure. A pressure bandage (not shown) is applied immediately post-operatively over the eyelids 24, 26 and removed after several days. The body 16 has a therapeutic shape with a volume that allows the cosmetic ophthalmic surgical conformer 10 to fit within the initial residual volume 30 and a shape that allows it to transmit the pressure created by the pressure bandage to the underlying tissues, e.g., conjunctiva 32, to aid in hemorrhage prevention and contouring of the underlying tissues.

After removal of the pressure bandage and with the eyelids 24, 26 parted, at least a portion of the cosmetic ophthalmic surgical conformer 10 and the at least one eye representation 14 is viewable by an observer. The cosmetic ophthalmic surgical conformer 10 can then be used until an artificial eye is available. While this depiction is for an enucleation procedure, it should be appreciated that the depiction would be similar for an evisceration or an exenteration procedure.

FIG. 6 shows a cross-sectional view of an artificial eye 36 positioned in the eye socket 22, depicted in FIG. 5, between the upper eyelid 24 and lower eyelid 26, and the implant 28 in a final residual volume 38. The final residual volume 38 is created when the swelling that defines the initial residual volume 30 subsides, see FIG. 5. Thus, the final residual volume 38 is larger in volume and of a different contour than the initial residual volume 30. Thus, an artificial eye 36 has a volume greater than the volume of the cosmetic ophthalmic surgical conformer 10 and has a different cross-sectional shape, as it is created from a mold (not shown) that duplicates the final residual volume 38. In particular, the artificial eye 36 has an outer surface 40 on which a mound 42 exists in a cornea region 44, which coincides with a cornea of an eyeball. The mound 42 gives the artificial eye 36 a non-constant thickness t′.

The standard placement of the cosmetic ophthalmic surgical conformer 10 in the eye socket 22 determines the nominal placement of the at least one eye representation 14. Referring to FIGS. 1, 5 and 6, it should be noted that the at least one eye representation 14 on the cosmetic ophthalmic surgical conformer 10 is positioned such that when the cosmetic ophthalmic surgical conformer 10 is placed in the eye socket 18 with at least a portion of its egg-shaped profile is visible to an observer (not shown), the at least one eye representation 14 is in a proximate location consistent with where that at least one eye representation will be on the artificial eye 36.

The cosmetic ophthalmic surgical conformer 10 is made from a suitable material, such as plastic or silicone, without vision correcting optical characteristics, such as those found in contact lenses. It is possible that portions of the ophthalmic surgical conformer could be transparent so that as much of the underlying tissue, e.g., conjunctiva, can be viewed therethrough as needed. It is understood, however, that the pupil and iris representations may block viewing of the underlying tissues. That said, the cosmetic ophthalmic surgical conformer 10 may be merely translucent or even opaque.

The material that makes up the cosmetic ophthalmic surgical conformer 10 should make the body 16 rigid or semi-rigid, so the cosmetic ophthalmic surgical conformer can transmit pressure. The material should therefore be generally non-compressible or non-deformable. The materials of the member 12 and the at least one eye representation 14, if not encapsulated in the member, should also be approved for use in a human body.

Referring to FIGS. 1-3 and 5, the member 12 and/or body 16 of the cosmetic ophthalmic surgical conformer 10 may be permeable, for example by having at least one hole 22 extending from the convex surface 18 to the concave surface 20. The permeability would permit medication (not shown) to pass through the cosmetic ophthalmic surgical conformer 10 to reach underlying tissues, such as the conjunctiva 32.

While there has been illustrated and described what is at present considered to be preferred and alternative embodiments of the claimed invention, it will be appreciated that numerous changes and modifications are likely to occur to those skilled in the art. It is intended in the appended claims to cover all those changes and modifications that fall within the spirit and scope of the claimed invention.

What is claimed is:

1. A cosmetic ophthalmic surgical conformer comprising:
a member and
at least one eye representation associated therewith, the combination defining a body consistent for use as an ophthalmic surgical conformer,
wherein when the body is properly positioned in an eye socket between the eyelids and an implant positioned in the eye socket, and the eyelids are parted the at least one representation is visible to an observer.

2. The cosmetic ophthalmic surgical conformer of claim 1 wherein the body has an egg-shaped profile.

3. The cosmetic ophthalmic surgical conformer of claim 2 wherein the at least one eye representation is positioned within the egg-shaped profile.

4. The cosmetic ophthalmic surgical conformer of claim 1 wherein the body has two opposed surfaces and there is a generally constant thickness therebetween.

5. The cosmetic ophthalmic surgical conformer of claim 4 wherein one opposed surface is concave.

6. The cosmetic ophthalmic surgical conformer of claim 1 wherein the cosmetic ophthalmic surgical conformer is made from a material that is at least semi-rigid.

7. The cosmetic ophthalmic surgical conformer of claim 1 wherein the body has a volume consistent with an initial residual volume of the eye socket into which it will be placed, the initial residual volume being smaller than a final residual volume of the eye socket into which an artificial eye will be placed, the artificial eye having a volume greater than the volume of the body.

8. The cosmetic ophthalmic surgical conformer of claim 1 wherein the body has a thickness that is substantially uniform.

9. The cosmetic ophthalmic surgical conformer of claim 8 wherein the body has a generally constant thickness between opposed convex and concave surfaces.

10. The cosmetic ophthalmic surgical conformer of claim 1 wherein the at least one eye representation is a pupil.

11. The cosmetic ophthalmic surgical conformer of claim 1 wherein the at least one eye representation is a sclera.

12. The cosmetic ophthalmic surgical conformer of claim 1 wherein the body is permeable.

13. The cosmetic ophthalmic surgical conformer of claim 1 having a volume for an application that is less than the volume of an eventual artificial eye for the application.

14. The cosmetic ophthalmic surgical conformer of claim 1 having a therapeutic shape for supporting a surgical procedure from the group consisting of evisceration, enucleation, and exenteration.

15. A method of using a cosmetic ophthalmic surgical conformer as a post-operative device prior to the fitting of an artificial eye comprising the steps of:
removing at least some of the structures of an eye creating a volume in an eye socket behind the eyelids,
positioning in a portion of the volume an implant; and
positioning immediately post-operatively a cosmetic ophthalmic surgical conformer having at least one representation of an eye in the eye socket between the eyelids and the implant such that the at least one representation is viewable by an observer.

16. The method of claim 15 wherein the step of removing at least some of the structures of an eye, the entire eye is removed revealing Tenon's capsule.

17. The method of claim 15 including the additional steps of:
placing an ophthalmic pressure dressing over the eyelids;
removing the pressure dressing, and
retaining the cosmetic ophthalmic surgical conformer in the eye socket.
